(19) United States
(12) Patent Application Publication
(10) Pub. No.: US 2026/0124021 A1
(43) Pub. Date: May 7, 2026

(54) MYOFUNCTIONAL ALIGNER

(71) Applicant: CODONIS AG, Muttenz (CH)

(72) Inventors: Fabio CIRILLO, Ormalingen (CH); Stefan STÜBINGER, Binningen (CH)

(21) Appl. No.: 18/855,371

(22) PCT Filed: Mar. 30, 2023
(86) PCT No.: PCT/EP2023/058354

§ 371 Date: Oct. 9, 2024

(30) Foreign Application Priority Data
Apr. 11, 2022 (DE) 10 2022 108 777.8

(51) Int. Cl. A61C 7/08; A61B 5/00; A61B 5/145

(52) U.S. Cl. CPC A61C 7/08; A61B 5/14507; A61B 5/6867; A61C 2201/00

(57) ABSTRACT

An aligner for orienting teeth of a set of teeth HAS a base part for specifying a preferred tooth position, the base part having a U-shape delimiting an arc-shaped interior space that is complementary to the U-shape. The aligner has a tongue anchor point directed from the base part towards the interior space for positioning, in the intended state, close to the palate of an oral cavity.

### FIELD OF THE INVENTION

Exemplary embodiments of the present invention relate to a myofunctional aligner as a treatment device in dentistry.

### BACKGROUND

Dental alignment devices in the form of special dental splints, so-called aligners, are known from the prior art. These aligners and the associated aligner therapy. Wherever fixed or removable orthodontic brackets and braces are rejected for aesthetic or oral hygiene reasons, these mostly transparent, thin-walled aligners are used. Unlike removable braces and brackets, these aligners should be changed every 1-4 weeks.

Aligners treat missing and crowded teeth in the early stages, preferably in the area of the front teeth. Aligners can also be used to close gaps, e.g., after orthodontic treatment.

The acceptance of aligners over braces, brackets, arches, or other methods among adolescents should also be particularly emphasized, as the aforementioned alternative methods of tooth correction have a strong impact on the visual appearance of the adolescent and/or their pronunciation.

Exemplary embodiments of the present invention are directed to extending the function of an aligner to include additional myofunctional stimulation.

### SUMMARY OF THE INVENTION

An aligner according to the invention is primarily used to align the teeth of a set of teeth. In a known manner, the aligner has a basic shape for specifying a preferred tooth position. This basic shape has a U-shape that delimits an arched inner space complementary to the U-shape. Due to the material, the basic shape can provide unintentional mechanical tongue stimulation, so that the tongue is stimulated to touch the aligner in the front area. This can result in incorrect pronunciation, e.g., lisping and the like, which can lead to speech therapy treatment after dental use, especially in children.

The aligner according to the invention therefore has a tongue anchor point directed from the basic shape in the direction of the inner space for its positioning in the intended state near the roof of the palate of an oral cavity.

The tongue anchor point is intended to stimulate the tongue, especially the tip of the tongue, so that the tongue is guided away from the basic shape towards the roof of the mouth. This encourages more tongue movement.

The aligner, in particular the basic shape, can advantageously have a U-shaped, in particular closed-walled, inner wall for lingual coverage of a row of teeth (upper or lower jaw).

Furthermore, the aligner, in particular the basic shape, can also have an outer wall for labial coverage of a row of teeth in the same way.

The aligner, in particular the basic shape, can advantageously have individual tooth contours with a predefined tooth position and with at least partially buccal overlapping areas of individual teeth of a row of teeth for optimum alignment.

Furthermore, the tongue anchor point can be designed as a protrusion that protrudes from the inner wall in the direction of the inner wall.

Alternatively, or additionally, the tongue anchor point can be designed as part of a bridging piece that extends from the first leg of the U-shape to the second leg of the U-shape and thus bridges the inner space.

The tongue anchor point can have a round body, in particular a ball and/or an ovoid.

The tongue anchor point can have a different surface texture compared to neighboring areas, in particular a grooving, corrugation, and/or nubbing. In particular, the neighboring areas are also part of the bridging piece or the protrusion.

The tongue anchor point can advantageously have a geometric figure, e.g., a bulge or nose, which protrudes at least partially in relation to a connection area of the protrusion and/or the bridging piece with the basic shape.

Furthermore, the protrusion or the bridging piece can be arranged to be detachable, preferably interchangeable, in particular latchable, in relation to the basic shape.

Alternatively, or additionally, the tongue anchor point can be arranged to be detachable, preferably interchangeable, in particular latchable, in relation to the protrusion or the bridging piece.

The tongue anchor point can preferably have a different surface haptic, preferably a different roughness and/or softness of the material, compared to neighboring areas of the bridging piece or the protrusion.

Furthermore, the tongue anchor point can have a chemical compound that is at least partially soluble in saliva, in particular a flavoring agent or an active substance. A stimulant other than the mechanical stimulant or an additional stimulant can be used here. The interchangeability of the tongue anchor point or the entire bridging piece or protrusion is particularly advantageous. An odorant, e.g., to improve breathing, is also conceivable.

The tongue anchor point can advantageously have a coating that is applied to a carrier material.

Advantageously, openings, in particular slots, can be provided in the area of the tongue anchor point.

A material of a different chemical composition can be arranged in the area of the tongue anchor point, or the tongue anchor point can consist of a different class of material in some areas or completely than the neighboring areas of the protrusion and/or the bridging piece. Another class of material is, for example, metal instead of plastic.

The tongue anchor point can advantageously have movable elements which are movable by the tongue.

The aligner, in particular the outer wall between the row of teeth and the lips, can have a shield segment for covering a row of teeth in certain areas, in particular the rows of teeth of the upper and lower jaw, wherein the shield segment has at least one protrusion, in particular an arrangement of several protrusions, for lip stimulation on the side facing away from the teeth.

The protrusions can preferably have a height of at least 0.1 mm, preferably greater than 0.5 mm. The protrusions can also preferably be cylindrical, spherical or frustoconical in shape.

In addition, the aligner can have sensors for determining your physical and/or chemical variable in the saliva and/or in the oral cavity, wherein the sensors are particularly preferably part of the protrusion or bridging piece.

The protrusions of the shield segment can also have sensor elements to monitor the progressive success of the training and/or the state of health in the mouth. The treatment device can also have actuators to support training.

Furthermore, the aligner may have actuators, in particular electrodes, for generating and/or transmitting stimulus signals, wherein the actuators are particularly preferably part of the protrusion or bridging piece.

Preferably, the aligner can be modularly adapted to different sized upper and lower jaw situations.

The aligner, in particular the bridging piece or the protrusion, may alternatively or additionally comprise an active substance and/or a flavoring agent and/or an indicator substance and/or a biomarker.

The aligner can also advantageously have a telescopic extension for adapting the aligner to the size of a set of teeth.

Alternatively, the aligner can also be designed in one piece, in particular monolithically.

Alternatively, aligners can have a modular design. Particularly preferably, the bridging piece and/or the protrusion with the tongue anchor point are interchangeably arranged as interchangeable modules on the basic shape. This allows the intensity of the stimulation to be adjusted. Every person reacts differently to mechanical stimulation or other forms of stimulation. Due to the interchangeability, the aligner can be individually adjusted.

### BRIEF DESCRIPTION OF THE DRAWINGS

In the following, the invention is described in more detail with reference to the drawing by means of exemplary embodiments, wherein further advantageous variants and designs are also discussed. It should be emphasized that the exemplary embodiments discussed below are not intended to describe the invention exhaustively, but that variants and equivalents not shown are also feasible and fall within the scope of the claims, wherein:

FIG. 1 shows a schematic diagram of a first variant of an aligner according to the invention;

FIG. 2 shows a schematic diagram of a second variant of an aligner according to the invention;

FIG. 3 shows a representation of the aligner in FIG. 2 with a bridging piece in the dismantled state;

FIG. 4a-t show variants for bridging piece FIGS. 2 and 3; and

FIG. 5 shows a variant for designing an outer wall 5 within the scope of the present invention.

### DETAILED DESCRIPTION

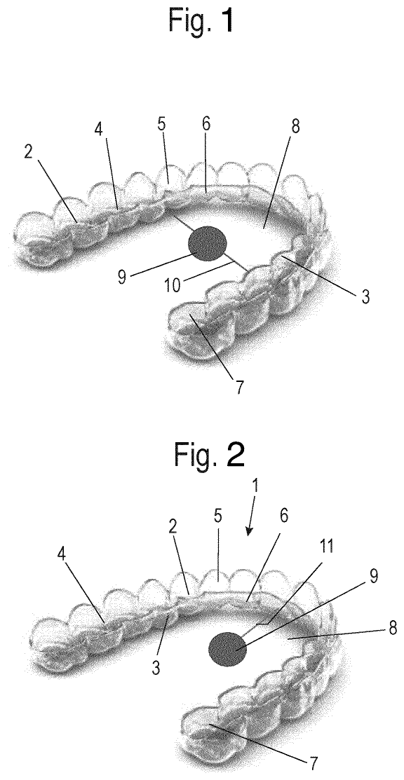

FIG. 1 shows a schematic representation of an aligner 1 for aligning teeth. The aligner 1 has a U-shaped basic shape 3 in plan view, with individual tooth contours 2 for better enclosure of individual teeth.

The sectional view perpendicular to the direction of a row of teeth through the aligner is also U-shaped, so that the aligner has a shell shape 7 to enclose the row of teeth.

An individual tooth contour 2 has partial areas and partially protrudes from the buccal and lingual overlapping areas of the teeth into the interdental spaces 4. The outer surface of the U-shaped leg of the basic shape forms a closed outer wall 5 and a closed inner wall 6 over the entire course of the U-shape of the basic body 3. The outer wall 5 and the inner wall 6 delimit the area for accommodating the teeth of the U-shaped shell form 7. The individual tooth contours 2 are formed depending on the type of tooth, e.g., canine, incisor, and/or molar.

Due to its U-shape, the inner wall 6 of the shell shape 7 defines an inner space 8 that is open beyond the molars and/or wisdom teeth and in which the user's tongue is typically positioned.

According to a first variant of the invention, a bridging piece 10 extends from the inner wall 6 into this inner space 8, in particular with a shape which is referred to below as the tongue anchor point 9.

In this way, the aligner takes into account a so-called myofunction, which, in addition to correcting the teeth, prevents lisping, mouth breathing, incorrect or missing swallowing patterns, hypersalivation, muscle spasms and/or muscle tension, insufficient lip and/or mouth closure, or other disorders.

Typically, the front third of the tongue behind the upper anterior teeth lies flat against the roof of the mouth in a normal set of teeth. The tongue anchor point 9 should be positioned at this point. The distance between the tongue anchor point 9 and the inner wall 6 should be designed accordingly.

The tongue anchor point encourages the patient to consciously place the tongue there. This applies to patients who are to have their teeth corrected and also to patients who need speech therapy or myofunctional therapy.

The aligner and the tray shape can be individually fabricated by the dental technician following an impression and/or scan, e.g., by the dentist.

In addition to the material of the tongue fix/anchor point 9 on the roof of the mouth, further elements can be provided, such as sensors, a modular structure, a drug release system and/or stimulation by means of nubs etc. on the outer wall 5 of the aligner.

The use of sensor elements has the special effect that, for example, when using biosensors, an inflammation status can be detected by the concentration of individual saliva ingredients. A conductivity measurement and/or other physical measurements, such as a temperature measurement, can be used to determine the oral cavity temperature, the moisture status in the interstitial space and, optionally, the extent of blood flow to the lips. In addition, pressure sensors can be used to measure the tongue pressure or the muscle tone of the masticatory and facial muscles, for example.

Electrodes can emit stimulation signals, such as stimulation current, to stimulate blood circulation and muscles. Actuators can also be used to massage lips, muscles or tissue in general.

In the case of electrically operated sensor elements or electrodes, an energy storage unit and/or a control and/or evaluation unit can be arranged inside the aligner, which is preferably encased in the material of the aligner and therefore does not come into contact with saliva. Due to the high mechanical load on electronic components when the aligner is used as intended, a multi-layer coating made of material of different hardnesses can also be provided.

The aligner 1 can be adapted to different sizes for different dentitions, i.e., children's teeth, mixed dentition (from milk teeth to permanent teeth), permanent teeth and dentures. Only the length of the shell shape 7 can be adjusted distally.

Since such sensors are active sensors that require power, the base body can also contain an energy source, energy cell, or energy harvesting device.

Preferably, these are passive electrochemical cells, thermoelectric elements, and/or induction-charged accumulators or capacitive electrical storage devices. Particularly in the case of elements that are arranged in the form of inlays on or in the aligner 1, these can be replaced and inserted into the aligner as required.

In addition, the sensors can be coupled directly with suitable external devices in order to address a control system, sound wave line and/or programs by means of sensor technology.

In addition, the external base station can be used to store the aligner, as well as to charge the aligner and/or read out the sensors. Furthermore, the external base station can transmit the data to a central storage, e.g., cloud storage and/or relay station, such as smartphones. Preferably, the external base station serves as a storage box, cleaning box and/or battery station while traveling.

FIG. 2 shows a second schematic variant of an embodiment of the invention according to the invention. Identical components and areas of the variant of FIG. 1 are provided with the same reference signs. The variant of FIG. 2 has a protrusion 11 instead of a bridging piece and an otherwise identical U-shaped basic shape 3. The protrusion 11 also serves to position a tongue anchor point 9 in the area of a roof of the mouth centrally in the inner space 8 of the U-shape of the basic shape.

This inner space 8 is formed by the basic shape 3, the adjacent row of teeth or an adjacent aligner, as well as the roof of the palate and the floor of the oral cavity when the mouth is closed and has an opening in the direction of the uvula.

The tongue anchor point 9 can be designed as an individual flat fixed/anchor point for the tongue, which can preferably be provided with a sensor, a suction cup, nubs or another surface texture, a tongue rest, a tongue stimulation point, e.g., a mechanical and/or gustatory stimulation.

The aligner can also be modularly expandable, e.g., for accommodating snoring splint additives, for releasing active substances, or also a patrix-matrix system for accommodating a small rubber ring that is placed on the tongue and must be docked, preferably in the area of the tongue anchor point 9.

The tongue anchor point achieves surface contact with the palate, particularly for the front third of the tongue, especially from the incisal papilla, which stimulates nasal breathing.

The lips are preferably in loose contact with the aligner. The chin muscle can relax and the rows of teeth are at rest. Overall, the resting position of the tongue is optimal.

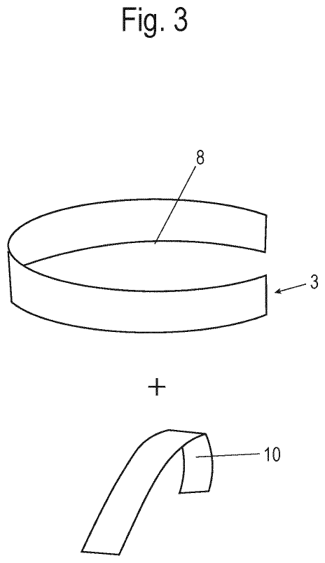

The bridging piece 10 of FIG. 1 is advantageously shaped in an arc in the direction of the roof of the palate, analogous to the variant shown in FIG. 3, so that the tongue anchor point 9 is brought closer to the roof of the palate.

The bridging piece 10 is shown in FIG. 3 as detachable, in particular replaceable. This can be achieved via any plug-in, clamp or snap-in connection or in another way, e.g., by using mechanical connecting means such as screws.

However, latching connections are preferred, as detachment of individual elements of the aligner can cause the risk of swallowing, especially during sleep.

Mechanical fastening means, on the other hand, often require a certain material thickness of the material in the connection area and increase the number of parts and thus the complexity of the aligner.

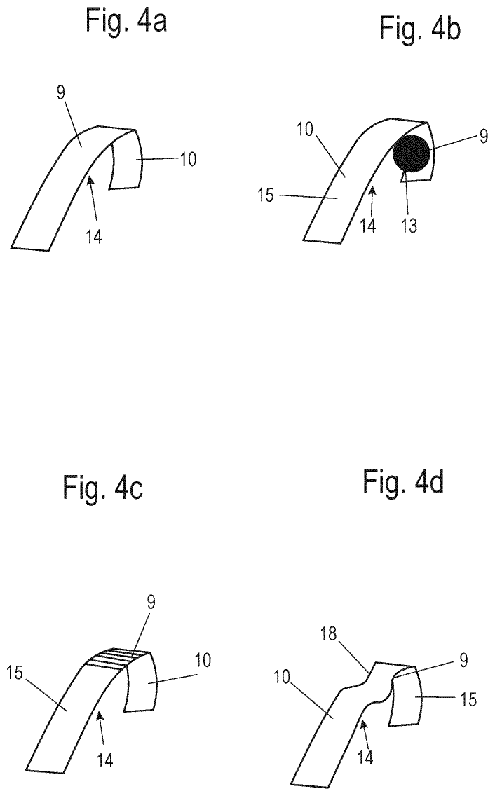

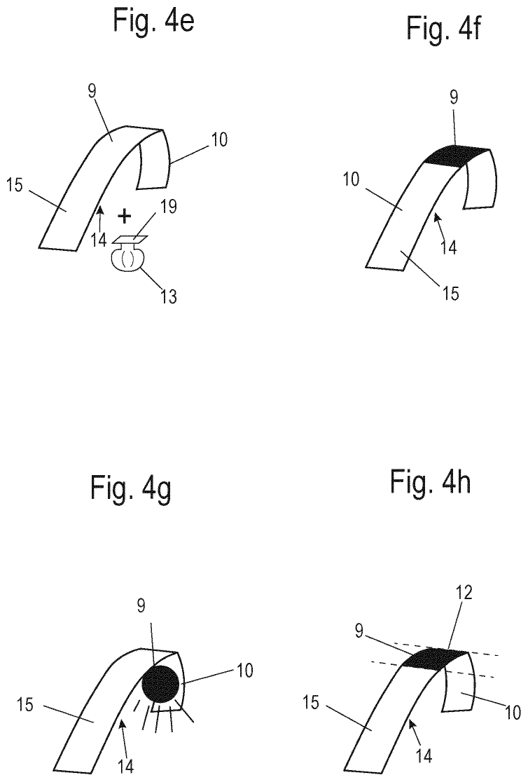

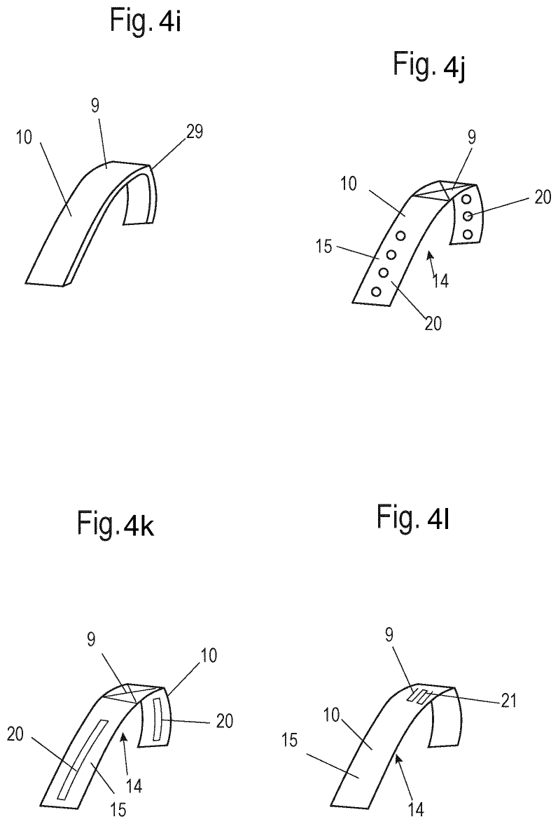

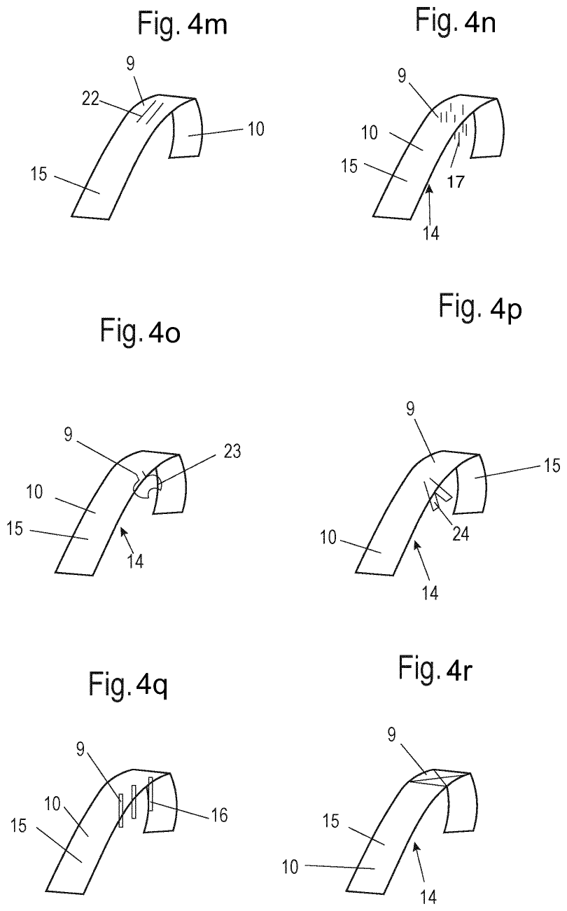

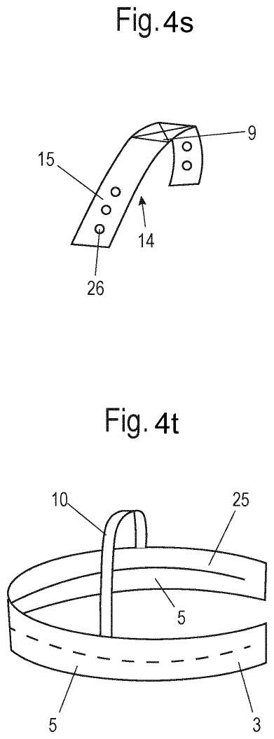

FIG. 4a first shows a bridging piece 10, which already contains the tongue anchor point 9 in the upper material area. The material of the bridging piece is uniform over the entire length of the bridging piece. In this respect, the tongue anchor point differs only in its position, but not in its physical or chemical properties, from the neighboring areas of the bridging piece, which serve to fix it to the base body of the aligner.

FIG. 4b shows a bridging piece with a ball 13 or an ovoid, which is arranged on the concave side 14 of the bridging piece 10, preferably in the middle. A sequence of several of these balls and/or ovoids can also be arranged along the concave side 14 of the bridging piece. The ball 13 or the ovoid or the sequence of several balls and/or ovoids can also be interchangeably arranged on the bridging piece, e.g. by means of a snap-in fastener or the like. Compared to the variant in FIG. 4a, these variants have a significantly stronger mechanical stimulation of the tongue.

The ball 13 or the ovoid or the aforementioned sequence of several of these elements can also be connected to the bridging piece 10 in such a way that the respective element is mechanically movable. For this purpose, either a mount can be provided which encloses the ball or the ovoid in certain areas. The same is also possible for the sequence of elements, similar to a curved roller bearing cage. Alternatively, the ovoid or the ball can also be provided with a central pin, which is fixed vertically to the bridging piece and to which the ball or ovoid is rotatably fixed.

FIG. 4c shows a variant in which the tongue anchor point 9 has a different surface texture, in the form of corrugation, compared to neighboring areas 15 of the bridging piece 10. Alternatively, for example, grooving and/or nubbing is also possible. An arrangement of thread-like or strip-like protruding elements 16 (see FIG. 4q), elements with fiber texture and/or flexible needles 17, e.g., made of rubber, (see FIG. 4r) or the like are also possible as a tongue anchor point 9 and increase the mechanical stimulation of the tongue.

FIG. 4d shows a variant in which the shape of the bridging piece 10 itself has been changed compared to the neighboring areas 15. For example, a concave bulge or nose 18 also protrudes into the concave side 14 of the bridging piece 10. This increases the mechanical stimulation of the tongue.

FIG. 4e shows a variant of a detachable connection of a ball 13 to the bridging piece 10, e.g. by means of a base 19. A complementary receiving opening with guide slots at the edges in the bridging piece 10 is not shown in FIG. 4e, but can easily be logically added so that the base can be inserted laterally into the bridging piece 10. Optionally, the guide slots can also have latching lugs, so that lateral displacement against the direction of insertion is no longer possible when the ball reaches the end position without actuating the latching mechanism for disengagement. The same naturally also applies to the connection of other geometric objects, e.g., an ovoid.

FIG. 4f shows a variant in which the tongue anchor point 9 has a different surface feel, preferably a different roughness and/or softness of the material, compared to neighboring areas 15 of the bridging piece 10. This can be a surface texture with greater roughness in the area of the tongue anchor point 9, but also a particularly smooth surface, i.e., with less roughness in the area of the tongue anchor point 9, or also a softer material in the area of the tongue anchor point, e.g., a crackling material, e.g., a crackling or rustling foil or a blister material or the like.

FIG. 4g shows a variant in which the tongue anchor point 9 releases a gustatory component, e.g., a sweetener, a bitter substance, a sour-tasting substance, or the like, in addition or as an alternative to the mechanical stimulant. Alternatively, or additionally, an active ingredient and/or an odorant, e.g., against bad breath, can also be released. The variation of FIG. 4g is not necessarily limited to a three-dimensional object, such as a sphere, but can also be a coating or the like.

FIG. 4h shows a further variant for attaching a tongue anchor point 9 to a bridging piece 10. Here in the variant as a clip or sleeve 12. The clip is open on one side and can be guided over the bridging piece by spreading it out and then clamped. The sleeve 12 can be slipped over the bridging piece by loosening it from the end side or from the side of the mechanical connection with the base body. Bayonet connections are also possible within the scope of the present invention for connecting the tongue anchor point to the bridging piece within the scope of the present invention.

FIG. 4i shows a variant of a bridging piece 10 with a tongue anchor point 9, wherein the tongue anchor point differs from neighboring areas only due to its position in the middle of the bridging piece 10, as in FIG. 4a. The bridging piece can have a single or multi-layer coating 29 or a lamination. Preferably, the coating or lamination may comprise an active ingredient and/or a flavoring agent and/or an odorant, which in particular is at least partially soluble in saliva and thus releasable. Alternatively, the bridging piece 10 can also be designed as a hollow element.

Surface roughening or smoothing in the area of the tongue anchor point 9, i.e. the central area of the bridging piece 10, can also be achieved by chemical or mechanical processing such as etching and/or grinding.

The areas 15 adjacent to the tongue anchor point 9 can also have various deposits, coatings, deposits, and/or inclusions or the like. For example, active ingredients or flavorings can be arranged in the adjacent areas. This is illustrated in FIGS. 4j and 4k. A corresponding depot 20 can be in the form of dots or strips. In FIGS. 4j and 4k, the tongue anchor point 9 is a haptically differentiated surface, e.g., in the form of a layer of lacquer.

In FIG. 4n, the bridging piece 10 has one or more strips 21 on the concave side of the bridging piece, at least in the area of the tongue anchor point 9. The strip or strips 21 have a different surface texture than the adjacent surfaces. The strip or strips 21 can also extend over the entire length of the bridging piece.

The bridging piece 10 of FIG. 4m has openings at least in the area of the tongue anchor point 9, in particular one or more slots 22 extending parallel to the longitudinal extension of the bridging piece 10.

In FIG. 4o, the bridging piece 10 has a tongue suction device 23 protruding from the concave side, which can also form part of a tongue anchor point 9.

Alternatively, a tongue support or tongue holding device 24, as shown in FIG. 4p, can also be formed as part of a tongue anchor point 9.

In FIG. 4r, a bridging piece 10 in the region of the tongue anchor point 9 can have a different surface area energy, a different hydrophobicity/hydrophilicity or a different electrical conductivity, in particular a metal surface and a polymer surface like the respective neighboring areas 15.

In FIG. 4s, the bridging piece has sensors 26 and/or actuators arranged in one or both areas adjacent to the tongue anchor point 9.

As can be seen from the aforementioned figures, a tongue anchor point 9 in the sense of the present application is to be understood in particular as an area and not only as a point in the strict mathematical sense.

As shown in FIG. 4t, a tooth whitening strip 25 can be arranged in the base body 3. In particular, this can be interchangeably arranged in the base body 3. Particularly preferably, the outer wall 5 or the inner wall 6 can have a recess arranged on the side inside the tooth, which allows a corresponding tooth whitening strip 25 to be accommodated.

The variants shown in FIGS. 4a-4t are only a few examples of the embodiment of the subject matter of the invention. Numerous other variants are possible within the scope of the present invention.

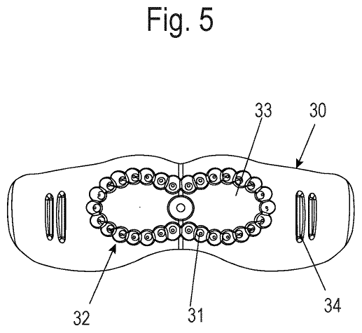

FIG. 5 shows a special design of an outer wall 5 of the basic shape 3 as a shield segment 30. The shield segment 30 can be arranged between the row of teeth and lips to cover a row of teeth in certain areas, in particular the rows of teeth of the upper and lower jaw. The shield segment 30 advantageously has at least one protrusion on the side facing away from the teeth, in particular an arrangement of several protrusions 31, for lip stimulation.

The protrusions 31 can preferably have a height of at least 0.8 mm, preferably greater than 1.0 mm. The protrusions can also preferably be cylindrical, spherical, or frustoconical in shape.

The protrusions 31 of the shield segment can also have sensor elements to monitor the progress of the training and/or the state of health in the mouth. The protrusions 31 can be arranged in such a way that they form a receptacle 32 with a receptacle area 33 in which an exchangeable sensor element or a sensor can be arranged.

Alternatively, or additionally, actuators, e.g., for additional electrical or mechanical lip stimulation, can also be used at the same point. At the edge of the protrusions 31 arranged in the central segment, the shield segment 30 has slots 34 for saliva drainage.

### REFERENCE SIGNS

1 Aligner
2 Individual tooth contours
3 U-shaped basic shape
4 Interdental spaces
5 Outer wall
6 Inner wall
7 Shell shape
8 Inner space
9 Tongue anchor point
10 Bridging piece
11 Protrusion
12 Sleeve
13 Ball
14 Concave side
15 Neighboring areas
16 Thread-like or strip-like protruding elements 16
17 Flexible needles
18 Concave bulge/nose
19 Base
20 Depot
21 Stripes
22 Slots
23 Tongue suction device
24 Tongue support or tongue holding device
25 Teeth whitening strip
26 Sensors
27 Shield segment
28 Protrusion
29 Coating
30 Shield segment
31 Protrusion
32 Receptacle
33 Receptacle area
34 Slots

1-19. (canceled)

20. An aligner configured to align teeth of a set of teeth, wherein the aligner comprises:
a basic shape specifying a preferred tooth position, wherein the basic shape has a U-shape delimiting an arched inner space complementary to the U-shape; and
a tongue anchor point directed from the basic shape in a direction of the arched inner space, wherein the tongue anchor point is configured for positioning in an intended state close to a roof of a palate of an oral cavity of the set of teeth.

21. The aligner of claim 20, wherein the basic shape, has an inner wall configured to lingually cover a row of teeth of the set of teeth.

22. The aligner of claim 21, wherein the basic shape, has an outer wall configured to labially cover the row of teeth of the set of teeth.

23. The aligner of claim 22, wherein the basic shape has individual tooth contours with a predefined tooth position and with at least partially buccal overlapping areas of individual teeth of a row of teeth of the set of teeth.

24. The aligner of claim 22, wherein the tongue anchor point is a protrusion protruding which protrudes from the inner wall in the direction of the inner wall.

25. The aligner of claim 20, wherein the tongue anchor point is part of a bridging piece extending from a first leg of the U-shape to a second leg of the U-shape and bridges the arched inner space.

26. The aligner of claim 20, wherein the tongue anchor point has a round body.

27. The aligner of claim 20, wherein the tongue anchor point has a different surface texture compared to areas of the aligner adjacent to the tongue anchor point.

28. The aligner of claim 20, wherein the tongue anchor point has a geometric figure protruding at least partially in relation to a connection region of a protrusion of the tongue anchor point or in relation to a connection region of a bridging piece of the tongue anchor point.

29. The aligner of claim 20, wherein at least the protrusion or the bridging piece is arranged detachably in relation to the basic shape.

30. The aligner of claim 20, wherein the tongue anchor point has a different surface haptic compared to neighboring areas of a bridging piece of the tongue anchor point or a protrusion of the tongue anchor point.

31. The aligner of claim 20, wherein the tongue anchor point has a chemical compound that is at least partially soluble in saliva.

32. The aligner of claim 20, wherein the tongue anchor point has a coating applied to a carrier material.

33. The aligner of claim 20, wherein openings are arranged in an area of the tongue anchor point.

34. The aligner of claim 20, wherein
a material of a different chemical composition is arranged in an area of the tongue anchor point, or
the tongue anchor point consists of a different class of material than areas neighboring a protrusion of the tongue anchor point or neighboring a bridging piece of the tongue anchor point.

35. The aligner of claim 20, wherein the tongue anchor point has movable elements which are movable by a tongue.

36. The aligner of claim 20, wherein the aligner has a shield segment configured to cover certain areas a row of teeth of the set of teeth, wherein the shield segment has at least one protrusion configured for lip stimulation on a side facing away from the teeth.

37. The aligner of claim 20, further comprising:
sensors configured to determine physical or a chemical variable in saliva or in an oral cavity.

38. The aligner of claim 20, further comprising:
actuators configured to generate and transmit stimulus signals.
